(19) United States
(12) Patent Application Publication
(10) Pub. No.: US 2022/0364076 A1
(43) Pub. Date: Nov. 17, 2022

(54) MICROFLUIDIC PLATFORM FOR SELECTIVE EXOSOME ISOLATION

(71) Applicant: The Texas A&M University System, College Station, TX (US)

(72) Inventors: Jun Kameoka, College Station, TX (US); Zheyuan Chen, College Station, TX (US)

(21) Appl. No.: 17/624,328

(22) PCT Filed: Jul. 2, 2020
(86) PCT No.: PCT/US20/40699

§ 371 Date: Dec. 31, 2021

Related U.S. Application Data
(60) Provisional application No. 62/870,033, filed on Jul. 2, 2019.

(51) Int. Cl. C12N 15/10; B01L 3/00

(52) U.S. Cl. CPC C12N 15/1017; B01L 3/502761; B01L 2200/0652; B01L 2300/0645; B01L 2300/069; B01L 2400/0421; B01L 2200/16

(57) ABSTRACT

The present disclosure pertains to a microfluidic platform. The microfluidic platform includes a top layer having a top inlet and outlet, a center layer having a center inlet and outlet, and a bottom layer having a bottom inlet and outlet. The microfluidic platform further includes a first porous membrane between the top and center layer, a second porous membrane between the center and bottom layer, a first electrode disposed on at least one of the top and bottom layers, and a second electrode disposed on at least one of the top and bottom layers. Additionally, the present disclosure pertains to a method for selective isolation. The method includes flowing a sample through a microfluidic platform, isolating a first component from the sample in a top layer, isolating a second component from the sample in a center layer, and isolating a third component from the sample in a bottom layer.

### CROSS-REFERENCE TO RELATED APPLICATIONS

This patent application claims priority from, and incorporates by reference the entire disclosure of, U.S. Provisional Application No. 62/870,033 filed on Jul. 2, 2019.

### TECHNICAL FIELD

The present disclosure relates generally to microfluidic platforms and more particularly, but not by way of limitation, to microfluidic platforms for selective exosome isolation and methods of use thereof.

### BACKGROUND

This section provides background information to facilitate a better understanding of the various aspects of the disclosure. It should be understood that the statements in this section of this document are to be read in this light, and not as admissions of prior art.

Exosomes are nano-sized extracellular vesicles (EVs) that play a role in cell-cell communication. Recently, there has been interest in exosome-related research, especially subgroups of exosomes as potential biomarkers for cancer diagnosis and prognosis. The present disclosure generally relates to new size-selective isolation methods via elastic lift force and nanomembrane filtration, and demonstrates liposome recovery rate of approximately 92.5% from a mixture solution of 1 μm polystyrene beads, 100 nm liposomes, and proteins for exosome isolation. The microfluidic platforms of the present disclosure offer an improved approach with short processing time (<2 hours) and low cost, and demonstrates broad applicability to biomarker studies, for example, early detection of cancer biomarker, diabetes studies. In addition, this platform can be used for separation of complex molecules or nanomaterials.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a microfluidic platform (e.g., an isolation platform). In some embodiments, the microfluidic platform includes a top layer having a top inlet and a top outlet, a center layer having a center inlet and a center outlet, a bottom layer having a bottom inlet and a bottom outlet, a first porous membrane between the top layer and the center layer, a second porous membrane between the center layer and the bottom layer, a first electrode disposed on at least one of the top layer and the bottom layer, and a second electrode disposed on at least one of the top layer and the bottom layer.

In another embodiment, the present disclosure pertains to a method for selective isolation. In some embodiments, the method includes flowing a sample through an isolation platform (e.g., a microfluidic platform). In some embodiments, the isolation platform includes a top layer having a top inlet and a top outlet, a center layer having a center inlet and a center outlet, a bottom layer having a bottom inlet and a bottom outlet, a first porous membrane between the top layer and the center layer, a second porous membrane between the center layer and the bottom layer, a first electrode disposed on at least one of the top layer and the bottom layer, and a second electrode disposed on at least one of the top layer and the bottom layer. In some embodiments, the method further includes isolating a first component from the sample in the top layer, where the first porous membrane precludes the first component from flowing into the center layer, isolating a second component from the sample in the center layer, where the second porous membrane precludes the second component from flowing into the bottom layer, and isolating a third component from the sample in the bottom layer.

In a further embodiment, the present disclosure pertains to a microfluidic platform (e.g., isolation platform) including a top layer having a top inlet and a top outlet, a plurality of center layers, each center layer of the plurality of center layers having a center inlet and a center outlet, a bottom layer having a bottom inlet and a bottom outlet, a first porous membrane between the top layer and a top center layer of the plurality of center layers, a plurality of center porous membranes between each center layer of the plurality of center layers, a second porous membrane between the bottom layer and a lowest center layer of the plurality of center layers, a first electrode disposed on at least one of the top layer and the bottom layer, and a second electrode disposed on at least one of the top layer and the bottom layer.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A illustrates a schematic diagram of a microfluidic device according to an aspect of the present disclosure.

FIG. 1B illustrates an assembly of the microfluidic device of FIG. 1A according to an aspect of the present disclosure.

FIG. 1C illustrates the microfluidic device of FIG. 1A and FIG. 1B including an electrode on the top of a microchannel of the top channel layer and the bottom of a microchannel on the bottom channel layer.

FIG. 2 illustrates a working schematic of a size-selective microfluidic platform for liposome isolation according to an aspect of the present disclosure.

FIG. 3 illustrates a size distribution for an initial solution and a solution isolated from a center channel outlet.

FIG. 4A illustrates a separation process at a top microchannel flowrate of 20 μl/min.

FIG. 4B illustrates particle concentration peaks of the top outlet and the top inlet at a top microchannel flowrate of 20 μl/min Flowrate is determined by the dimensions.

FIG. 5A illustrates a separation process at a top microchannel flowrate of 10 μl/min.

FIG. 5B illustrates particle concentration peaks of the center outlet, the top outlet, and the top inlet at a top microchannel flowrate of 10 μl/min Flowrate is determined by the dimensions.

FIG. 6A illustrates a separation process at a top microchannel flowrate of 5 μl/min.

FIG. 6B illustrates particle concentration peaks of the center outlet and the top inlet at a top microchannel flowrate of 5 μl/min Flowrate is determined by the dimensions.

FIG. 7 illustrates electrical field effects of particle concentration.

FIG. 8 illustrates size-selectivity via particle concentration for a microfluidic device at a center outlet and particle concentration at a top inlet.

FIG. 9 illustrates a schematic diagram of a microfluidic platform capable to isolate and pre-concentrate exosomes utilizing a hydrogel according to aspects of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Extracellular vesicles (EVs) are heterogenous biological particles with lipid bilayer membranous structures that hold information in the form of nucleic acids, proteins, or lipids, hence, acting as vehicles of intracellular communication. They facilitate normal cell homeostasis via the exchange of biological materials across cells. Moreover, they package unique molecular and lipid constituents depending upon their cell of origin and the biophysical properties of vesicles. Recently, “exosomes”, a nanoscale subpopulation of EVs, have attracted intense interest. Exosomes have great advantages as a promising source of cancer biomarkers for diagnosis and prognostic. They can be collected without invasion, making it possible for continuous monitoring of disease progression and prognosis as liquid biopsies. Exosomes are abundant and are included in most body fluids such as, but not limited to, blood, saliva, urine, lymph, and milk.

To investigate exosomes as potential diagnostic and prognosis biomarker candidates in liquid biopsies, various isolation methods have been developed. Currently, ultracentrifugation has been a most commonly used technique for exosome isolation, which typically requires a sequence of centrifugation steps eventually reaching rotation speeds of up to 200000 G. However, this approach is time-consuming and provides low exosome recovery, low purity, and poor reproducibility due to the presence of non-exosomal proteins and microvesicular debris. Moreover, there is evidence that the high centrifugal force (100,000 G to 200,000 G) can cause exosome fusion, coagulation, and may damage their structures, properties, and functions. In addition, it requires well-trained personnel to operate and is quite costly. Other methods including precipitation, immunoaffinity capture, acoustic-wave based separation, nanoscale lateral displacement arrays, nanowire trapping, membrane filtration system, and dielectrophoretic separation, have also been implemented. However, these methods are limited by drawbacks, such as, for example, additional reagents and/or labels, pre-treatment steps, long processing time, low exosome recovery, and low purity.

As such, aspects of the present disclosure are directed towards microfluidic platforms (e.g., isolation platforms) that can consistently separate exosomes based on size via elastic lift force and nanomembrane filtrations. The platforms and methods, as disclosed herein, provide significant advantages in identifying and harvesting size specific exosomes, such as, but not limited to, short processing time (less than 2 hours), low cost, high recovery rate (approximately 94%), high recovery volume, and high purity. The platforms discussed in further detail herein, improve exosome biomarker studies and open new avenues for precision medicine for cancer prognosis and early detection.

The platforms of the present disclosure can be fabricated from, for example, polydimethylsiloxane (PDMS) or polymer or paper substrates based multi-layered microfluidic platforms, or other similar materials. The platforms, in some embodiments, can have five horizontally aligned microchannels and four nanomembrane filters (porous nanomembranes) with four pore sizes (e.g., 120 nm, 80 nm, 50 nm, and 30 nm) sandwiched between microchannels. In some embodiments, the microfluidic platform can have three horizontally aligned microchannels and two nanomembrane filters with two pore sizes (e.g., 200 nm and 30 nm) sandwiched between these microchannels. In some embodiments, the microfluidic platform can have three horizontally aligned microchannels and two nanomembrane filters with two pore sizes (e.g., 100 nm and 30 nm) sandwiched between these microchannels. In some embodiments, the microfluidic platform can have more microchannels or nanomembrane filters to provide a higher resolution of separation. Gold, or other metallic electrodes, are patterned on the top and bottom of various microchannels to induce elastic lift force through the nanomembrane filters. Exosomes are then separated based on membrane pore sizes.

In one aspect, an aim of the present disclosure is to provide for a microfluidic platform to isolate exosomes based on size, which is a new methodology to isolate exosomes. To this end, the present disclosure discusses new fabrication processes to construct microfluidic platforms (e.g., isolation platforms) as disclosed herein. Additionally, the platforms and methods disclosed herein can isolate exosomes based on size (size selective isolation) with high resolution, which assists in determining exosome biomarkers. Disclosed herein is an approach that provides for a considerably quicker exosome separation method than that of conventional exosome separation approaches. Furthermore, the isolated exosome recovery rate is considerably higher than that of conventional exosome separation methods.

In some embodiments, the fabrication processes disclosed herein are improved to provide a much simpler fabrication, which can ultimately further reduce the cost of manufacture. In some embodiments, various pumping systems can be miniaturized such that macro-pump systems can be eliminated. This can allow for a more user-friendly and widely accepted platform. Furthermore, the platforms and methods as disclosed herein provide potential for clinical applications. Current exosome separation methods, as previously discussed above, involve centrifugation, immune affinity, membrane, and nanostructures. However, these methods fail to provide for quick isolation of exosomes, high isolation recovery rate, and low cost. Additionally, proteins can be eliminated from the samples, and, as a result of the microfluidic designs and methods of use, there is less membrane fouling in samples.

As such, disclosed herein a microfluidic platform and method of use that demonstrates the isolation of exosomes within two hours in cancer cell culture media. The separation recovery is approximately 94.2%. Additionally, the microfluidic platforms and methods of use thereof, also isolate liposomes from other particles. These platforms and methods of use are capable of isolating exosomes in patient samples that can include, for example, blood or urine, for early detection of cancer or cancer prognosis. In addition, RNA and microRNA in exosomes can be isolated, which provide potential biomarkers for various diseases including, but not limited to, cancer, diabetes, autoimmune diseases, Parkinson's disease, Alzheimer's, or combinations of the same and like. The platforms and methods of use disclosed herein are capable of exploiting various volumes for isolation. For example, higher or lower volumes can be utilized for the isolation of RNA, microRNA, exosomes, liposomes, or combinations of the same and like.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

In view of the foregoing, presented herein is a unique platform targeting a consistently enhanced isolation method based on a size-selective process utilizing elastic lift force and nanomembrane filtration. As a result, the processes disclosed herein enable identifying and harvesting size specific exosomes with additional advantages of, for example, having short processing time and lower costs relative to conventions methods.

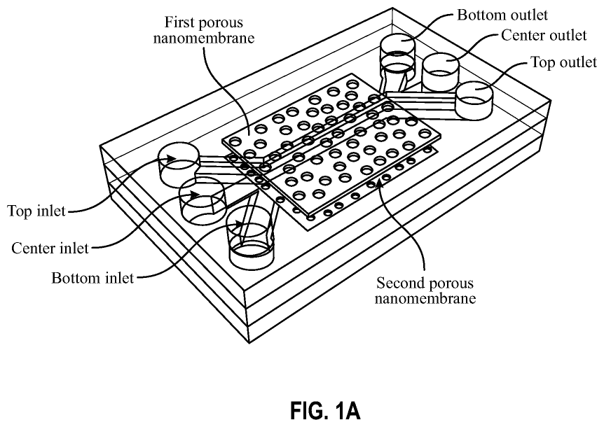

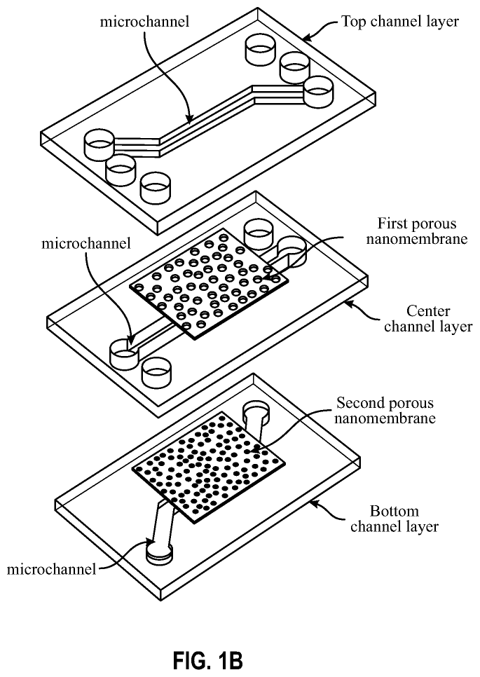

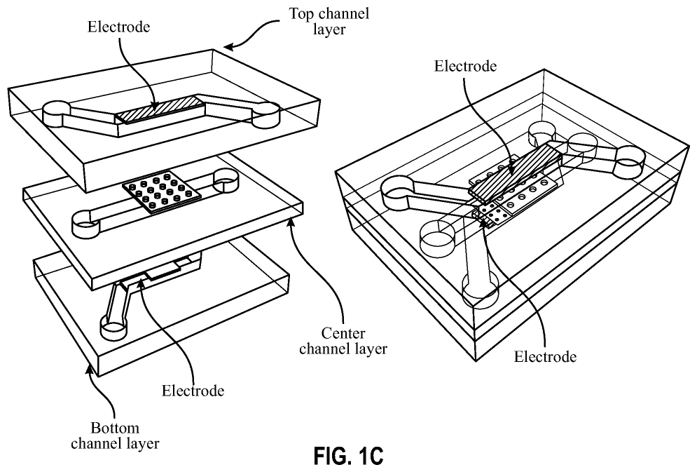

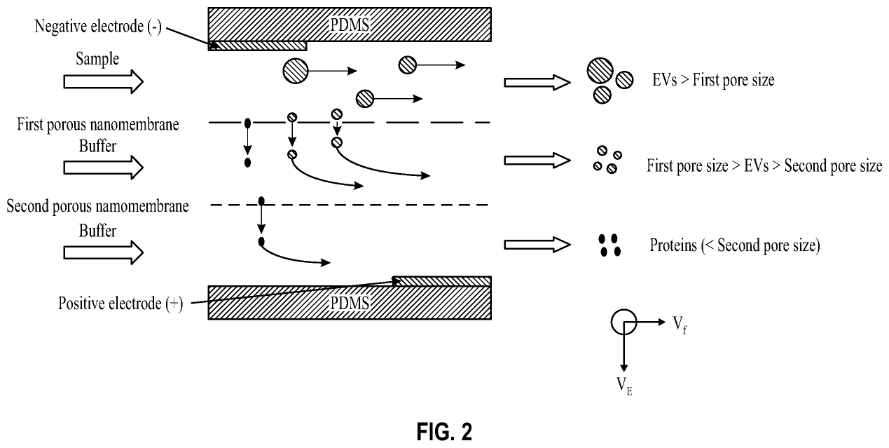

In an embodiment, as illustrated in FIG. 1A-1B, a microfluidic platform has three horizontally aligned microchannels (formed in a top, center, and bottom channel layer) and two porous nanomembranes (first and second porous nanomembranes) sandwiched between the microchannels. The first and second porous nanomembranes have a first pore size and a second pore size, respectively. As illustrated in FIG. 1C, electrodes, such as, for example, gold electrodes are connected to the top and bottom of the microchannels of the top channel layer and the bottom channel layer, respectively, to induce force through the two porous nanomembranes. As shown in FIG. 2, and described with respect to FIG. 1A-FIG. 1C, particles are separated based on the pore sizes of the porous nanomembranes (first and second pore sizes). Due to the elastic force between the top and bottom microchannels, proteins and particles less than the first pore size of the first porous nanomembrane are forced to pass through the first porous nanomembrane and are transported into the center channel. Particles larger than the first pore size of the first porous nanomembrane are separated from particles less than the first pore size of the first porous nanomembrane and are guided to the top outlet. Particles that are smaller than the first pore size of the first porous nanomembrane and larger than the second pore size of the second porous nanomembrane are guided to the center outlet. Particles smaller than the second pore size of the second porous nanomembrane, are guided into the bottom channel through the second porous nanomembrane and to the bottom outlet.

As an example, the microfluidic platform can have three or more horizontally aligned microchannels and two porous nanomembranes with pore sizes of 100 nm and 30 nm sandwiched between the microchannels. Gold electrodes can be connected to the top and bottom of the microchannels of the top channel layer and the bottom channel layer, respectively, to induce force through the two porous nanomembranes. In this example, proteins and particles less than 100 nm in size are forced to pass through the 100 nm porous nanomembrane and are transported into the center channel Particles larger than 100 nm are separated from particles less than 100 nm and are guided to the top outlet. Proteins, for example, are quickly guided into the bottom channel through the 30 nm porous nanomembrane.

A sample solutions containing 1 μm diameter polystyrene beads (representative of large particles), 100 nm diameter liposomes (representative of exosomes), and antibodies (<10 nm, representative of proteins) are used to validate the feasibility of the isolation process and evaluate the separation efficacy.

The velocity (v) can be calculated based on Equation 1, shown below, in laminar flow conditions.

$$v = \frac{Q E}{6 π μ r} \qquad Equation 1$$

In Equation 1 above, “Q”, “E”, “μ”, and “r” are particle charge, applied electrostatic field, viscosity of fluid, and particle radius, respectively. The most dominant effect comes from the hydrodynamic radii of the proteins (<10 nm) and liposomes (100 nm). Particles of smaller dimension tend to move faster in vertical direction. The elastic lift velocity is therefore inversely proportional to the radius of the particle. The vertical elastic lift velocity of the larger particles is much slower than those of the smaller particles and proteins, thereby enabling the proteins and smaller dimensions of liposomes to pass through nanomembranes vertically.

Device Fabrication and Assembly. As shown in FIG. 1A, the microfluidic platform has three polydimethylsiloxane (PDMS) microchannels (200 μm in height and 1000 μm in width) and two track-etched polycarbonate (PC) porous nanomembranes (Whatman Nuclepore, Sigma Aldrich) with pore size of 200 nm and 30 nm, corresponding to the first and second porous nanomembranes, respectively (200 nm/30 nm microfluidic device). The microfluidic channels were prepared by soft lithography with some modifications. In brief, SU-8 2075 photoresist (MicroChem) was spin-coated on a 3-inch silicon wafer. The SU-8 photoresist was patterned by photolithographic process as the top, center, and bottom channel layer (microchannel layers) master molds. A PDMS solution was prepared by mixing original PDMS solution with cross-linker solution (Sylgard 184, Corning) at a ratio of 10 to 1. The well-mixed PDMS solution was then dispensed on these molds. Upon curing at 65° C. for 4 hours, these PDMS channel layers were peeled off from the molds. Three inlets and outlets holes were formed in the top channel layer, and the center channel layer was punched with two vials for connecting the inlet and outlet of the bottom microchannel A large through-hole was also punched at the center of the center channel layer.

Then, these PDMS channel layers were treated with oxygen plasma for 2 minutes. The 30 nm porous PC nanomembrane was sandwiched by the bottom and center channel layers (FIG. 1B). The center-bottom device was cured at 65° C. for 2 hours to improve bonding quality. With the similar molding and bonding process, the 200 nm porous PC nanomembrane was also sandwiched by the top and center-bottom layers (FIG. 1B). The three-layer device was baked at 65° C. for 2 hours. After bonding process, six pieces of tubing (Tygon 3350) were connected to the inlets and outlets of the device. The free ends of the inlet tubing were connected to syringes, while the free ends of the outlet tubing were connected to containers that collect the separated solutions. Gold wires were connected to the top inlet tubing and bottom outlet tubing as electrodes (FIG. 1C).

Sample Preparation and Experiment Set-up. The stock solution of polystyrene beads stock solution was purchased from Sigma Aldrich. The mean particle size of polystyrene beads is 1 μm. The fluorescent liposome stock solution was purchased from FormuMax. The excitation wavelength is at 496 nm and the emission wavelength is at 519 nm. The mean particle diameter of liposomes is 100 nm with a range of 90 nm to 120 nm. The goat anti-mouse immunoglobulin G (IgG) secondary antibody stock solution was purchased from Life Technologies with a concentration of 2 mg/ml. The antibody was used to represent proteins.

The initial solution was prepared by mixing 1 μl the liposome stock solution, 1 μl of the antibody (IgG) stock solution, and 1 μl of the polystyrene beads stock solution with phosphate-buffered saline (PBS) solution added to a total volume of 1 ml. 0.5 ml of the initial solution was pumped into the top microchannel at a flow rate of 5 μl/min, and the PBS buffer solutions were pumped into the center and bottom microchannels at flow rates of 10 and 20 μl/min, respectively. The applied voltage was kept at 200 V. The total processing time for isolation was less than 2 hours. The rest of the initial solution and isolated solutions were kept in the refrigerator at 4° C. for the following quantification analysis.

Characterization and Analysis. The scanning electron microscope (SEM) images of the PC membranes were taken by Tescan FERA-3. A 5-nm-thick palladium thin film was deposited by sputtering on the surface of the sample before loading into the chamber of the equipment. A Nikon Eclipse Ti microscope with a Zyla 4.2 camera (Andor) was used for fluorescence imaging. The images were captured by NIS Elements Advanced Research 4.30.02 software.

The size distribution and total numbers of particles in the initial and isolated solutions were measured by nanoparticle tracking analysis (NTA). The measurement was conducted at room temperature using a NanoSight LM10 system with emitting laser λ=405 nm (Malvern). Before testing, the original sample solution and the isolated solutions were diluted 100 times in a concentration range of 107 to 109 particles per microliter. Then, solution was introduced by a syringe manually and the video images were recorded for 60 seconds using NTA software version 3.2. Each experiment was repeated at least three times. The diameters of the particles were also studied by dynamic light scattering (DLS) apparatus (Zetasizer nano, Malvern).

Similar experimentation was performed via a microfluidic platform that utilized three PDMS microchannels and two track-etched PC porous nanomembranes with pore size of 100 nm and 30 nm, corresponding to the first and second porous nanomembranes, respectively (100 nm/30 nm microfluidic device).

### Results and Discussion

Fluorescent Image. During the separation process (FIG. 2), fluorescence images were taken at the center outlet of the device. With respect to the 200 nm/30 nm microfluidic device, from the fluorescent microscopic images, it was concluded that the 1,2-dihexadecanoyl-sn-glycero-3-phosphoethanolamine (DHPE) labeled liposomes were continuously guided to the center outlet. When the initial solutions were pumped into the top channel, liposomes passed through the 200 nm nanomembrane to the center channel due to elastic lift force and flowed to the center outlet.

DLS Analysis. The initial solution and the isolated solutions were collected and then diluted by PBS solution to align with testing requirements for DLS measurement. With respect to the 200 nm/30 nm microfluidic device, the initial solution that was pumped into the top inlet had two obvious peaks at about 100 nm and 1 μm, which contained mainly liposomes and polystyrene beads, respectively. No obvious peak in the range of 1 to 10 nm was detected although the IgG antibody was included in the initial solution. This was because the initial solution was polydisperse and the intensity of the IgG antibody was not strong enough to be detected compared to particles with larger diameters. The DLS analysis of the center isolated solution demonstrated a visible peak at around 100 nm, which contained mainly liposomes. No peaks were detected at around 10 nm and 1 μm. Meanwhile, the particle distribution graph from the bottom isolated solution demonstrated a peak at around several nanometers, which contained proteins (IgG) mainly. No peaks were detected at around 100 nm and 1 μm. Thus, the results suggested that the liposomes in the initial solution were successfully separated from the mixture solution and guided to the center microchannel. The IgG antibodies, representative of proteins, were also separated and guided to the bottom microchannel.

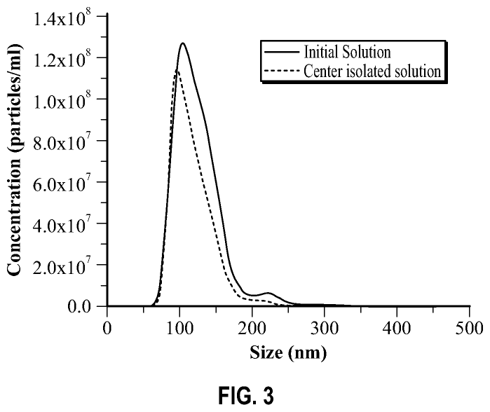

NTA Analysis. The size distribution and total numbers of liposomes in the initial solution and isolated solution in the center outlet were analyzed by NTA. With respect to the 200 nm/30 nm microfluidic device, and illustrated in FIG. 3, liposomes in both the initial and center isolated solutions showed similar size distribution profiles. Peaks were identified at around 250 nm, and could be aggregations of multiple liposomes. The center isolated solution exhibited a size distribution with a single peak at 98 nm. The initial solution showed a broad size distribution with a single peak at 106 nm, which represented a slight shift from the one acquired from the center outlet. This difference could be attributed to the resolution limits of NTA when testing highly heterogeneous samples.

NTA was further used for qualification of liposome concentrations. The volume of the center solution collected was twice of that of the initial solution. Liposome concentration in the center outlet was corrected by multiplying the dilution factor (2). The total numbers of liposomes (90 nm to 120 nm) from the center outlet was calculated at 3.12×10⁹ per microliter. The total numbers of liposomes (90 nm to 120 nm) in the initial solution was 3.37×10⁹ per microliter. The recovery rate is defined as the fraction of the total numbers of isolated liposomes and the total numbers of liposomes in the initial solution. Overall, the present microfluidic platform showed a high recovery rate of 92.5% for liposome isolation.

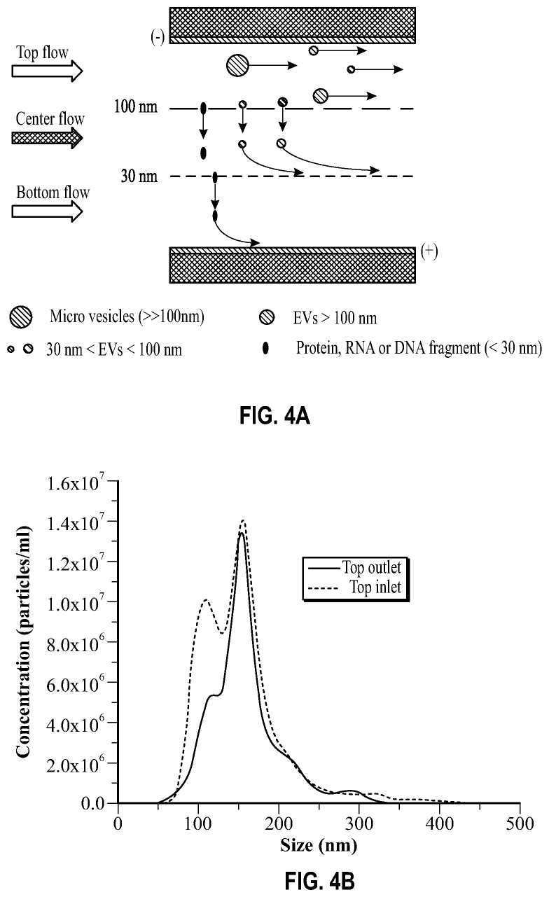

NTA was additionally used for qualification of liposome concentrations at varying top microchannel flowrates for the 100 nm/30 nm microfluidic device. FIG. 4A illustrates the separation process at a top microchannel flowrate of 20 μl/min. As shown in FIG. 4A, a top microchannel flowrate of 20 μl/min was too fast to completely separate a majority of particles less than 100 nm. This is evidenced in FIG. 4B, illustrating the particle concentration peaks of the top outlet and the top inlet.

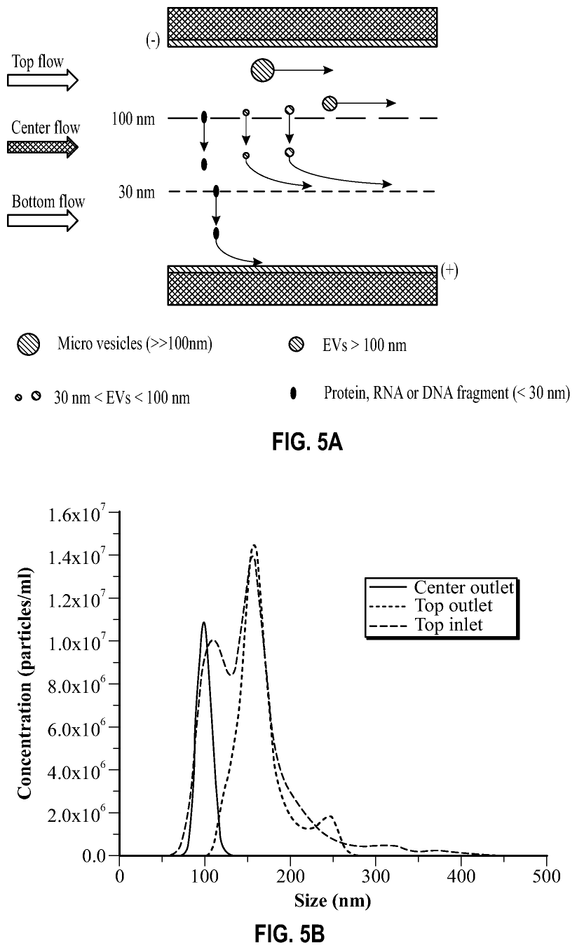

FIG. 5A illustrates the separation process at a top microchannel flowrate of 10 μl/min. As indicated in FIG. 5A, upon lowering the top microchannel flowrate, separation occurred between particles greater than and much greater than 100 nm, such as micro vesicles and extracellular vesicles (EVs) greater than 100 nm, respectively, by preclusion at the first porous nanomembrane. The second porous nanomembrane separated particles less than 30 nm, representative of, for example, protein, RNA, or DNA fragments, from EVs less than 100 nm, but greater than 30 nm. This is evidenced in FIG. 5B, illustrating the concentrations at the center outlet, the top outlet, and the top inlet. Data is summarized in Table 1, shown below.

TABLE 1

| Particles (nm) | Center Outlet | Top Inlet | Recovery Rate | Purity Yield |
|---|---|---|---|---|
| 30-100 | 1.29 × 10¹⁰ | 1.37 × 10¹⁰ | 94.2% | 56.3% |
| 30-110 | 2.05 × 10¹⁰ | 2.36 × 10¹⁰ | 90.7% | 86.9% |
| 30-120 | 2.24 × 10¹⁰ | 3.33 × 10¹⁰ | 67.3% | 99.1% |
| 30-150 | 2.25 × 10¹⁰ | 6.26 × 10¹⁰ | 35.9% | 99.6% |
| Total | 2.26 × 10¹⁰ | 1.17 × 10¹¹ | | |

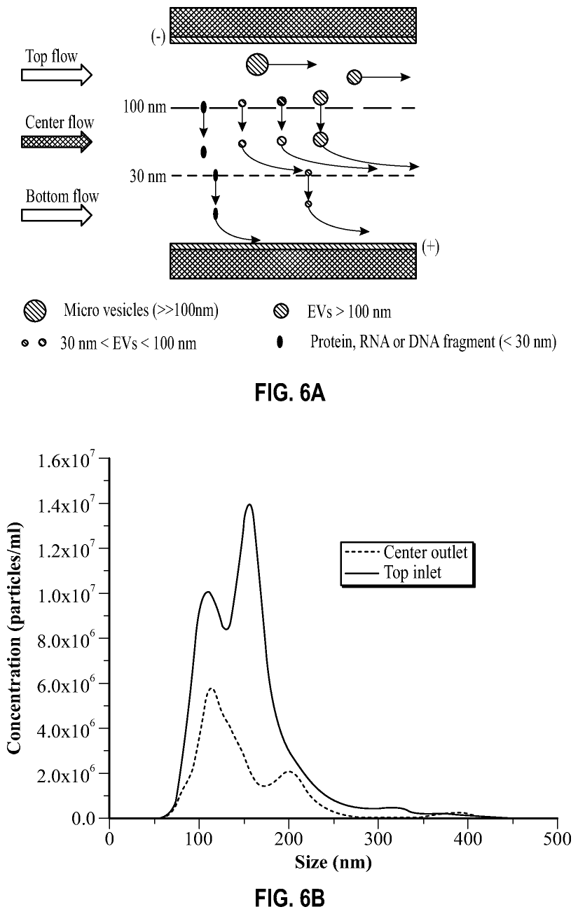

FIG. 6A illustrates the separation process at a top microchannel flowrate of 5 μl/min. As indicated in FIG. 6A, upon lowering the top microchannel flowrate to 5 μl/min, separation occurred between particles greater than and much greater than 100 nm, such as micro vesicles and EVs greater than 100 nm, respectively, at the first porous nanomembrane. However, some particles greater than 100 nm also passed through the first porous nanomembrane. The second porous nanomembrane separated particles less than 30 nm, representative of, for example, protein, RNA, or DNA fragments, from particles less than 100 nm, but greater than 30 nm. However, some particles greater than 30 nm and less than 100 nm also passed through the second porous nanomembrane. This is evidenced in FIG. 6B, illustrating the concentrations at the center outlet and the top inlet. Data is summarized in Table 2, shown below.

TABLE 2

| Particles (nm) | Center Outlet | Top Inlet | Recovery Rate | Purity Yield |
|---|---|---|---|---|
| 30-100 | 6.58 × 10⁹ | 1.37 × 10¹⁰ | 48.0% | 15.0% |
| 30-110 | 1.19 × 10¹⁰ | 2.36 × 10¹⁰ | 50.4% | 27.2% |
| 30-120 | 1.73 × 10¹⁰ | 3.33 × 10¹⁰ | 52.0% | 39.5% |
| 30-150 | 2.83 × 10¹⁰ | 6.26 × 10¹⁰ | 45.2% | 64.6% |
| Total | 4.38 × 10¹⁰ | 1.17 × 10¹¹ | | |

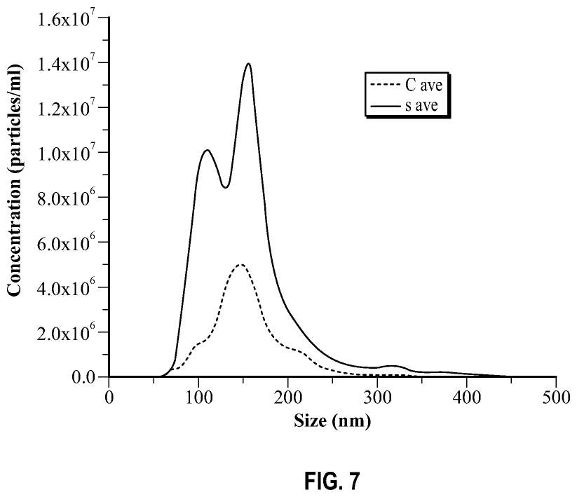

Additional Analysis. FIG. 7 illustrates electrical field effects of particle concentration. Electric field is a factor utilized to demonstrate particle separation. For instance, if the electric field is too strong, as illustrated in ave c of FIG. 7, particles are forced through the porous nanomembrane to reach the bottom channel. However, when appropriate electric field is applied, particles can be isolated in the middle channel.

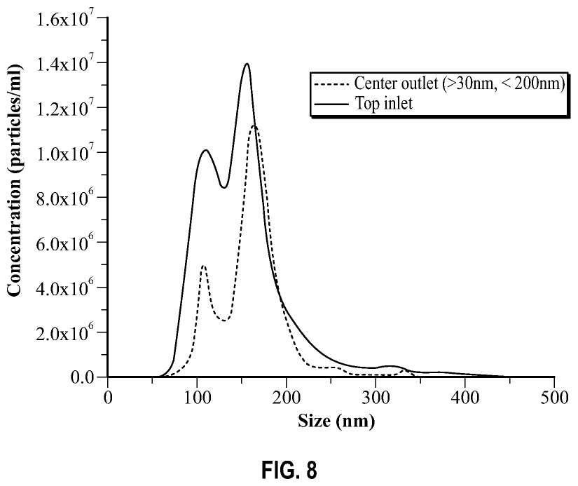

FIG. 8 illustrates size-selectivity via particle concentration for the 200 nm/30 nm microfluidic device at the center outlet (i.e., greater than 30 nm and less 200 nm particle sizes) and particle concentration at the top inlet. Data is summarized in Table 3, shown below.

TABLE 3

| Particles (nm) | Center Outlet | Top Inlet | Recovery Rate |
|---|---|---|---|
| 30-100 | 2.4 × 10⁹ | 1.37 × 10¹⁰ | 17.5% |
| 30-200 | 5.8 × 10¹⁰ | | |
| Total | 6.5 × 10¹⁰ | 1.14 × 10¹¹ | |

MicroRNA Extraction. The microfluidic platforms (e.g., isolation platforms) disclosed herein can be designed to isolate and pre-concentrate exosomes to ultimately extract micro RNA. The preceding demonstrated the isolation of exosomes based on their dimensions with porous nanomembranes and electrophoresis resulting in high purity and reproducibility. In these new microfluidic platforms, the functions of the pre-concentration and lysing exosomes, and microRNA extraction can be integrated in the microfluidic platforms.

As previously discussed, current exosome isolation techniques include conventional ultracentrifugation, membrane filtration (size-based separations), and immune particle isolation (immune separations). However, these systems encounter multiple challenges such as low reproducibility (size based separation), low specificity (size based separation) and low yield (immune separation), long processing times, and high cost. Because of these limitations, the development of new reproducible exosome isolation methods from patient samples are desirable to advance exosome studies. The proposed microfluidic platforms herein target improvements to the exosome isolation yield and specificity via viscoelastic lift force exosome guiding, nanomembrane filtering, and pre-concentration of exosomes via hydrogel nanostructures.

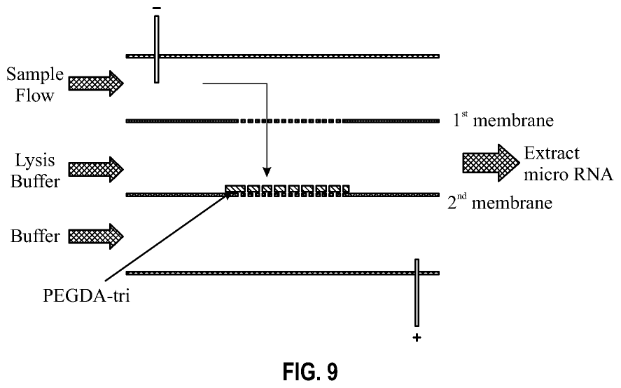

FIG. 9 illustrates a schematic diagram of a microfluidic platform capable to isolate and pre-concentrate exosomes. The microfluidic platform utilizes a hydrogel composed of polyethylene glycol dimethacrylate (PEGDA)-tri that has a PEGDA backboard and amine group at the molecular terminal, with an under pH 7 range. The surface of the hydrogel is positively charged such that the surface can attract negatively charged exosomes.

Exosomes are isolated by the first nanomembrane based on the dimensions of the exosomes (e.g., less than 150 nm), then exosomes less than 150 nm and proteins are transported into the second channel via an electrostatic field. Exosomes and proteins are continuously attracted to the third channel. The porous nanomembrane (30 nm porosity) is integrated with a polyethylene glycol dimethacrylate-triamine (PEGDA-triA) hydrogel and is placed between the second and third channel layers to filter proteins. This integrated porous nanomembrane has a positive surface potential to attract negatively charged molecules. As a result, size-isolated exosomes are trapped (exosomes are negatively charged) at the hydrogel porous nanomembrane. To extract microRNA in exosome, an exosome lysis buffer is pumped into the second channel MicroRNA is also negatively charged, so high flow of lysis buffer prevents the trapping microRNA at the PEGDA-trA hydrogel.

### CONCLUSION

The present disclosure illustrates a conclusively developed and demonstrated size-selective liposome isolation platform. Polystyrene beads (representative of large particles), liposomes (representative of exosomes), and IgG antibody (representative of proteins) in the initial solutions were successfully separated with a liposome recovery rate of up to 92.5%, with some microfluidic platforms exhibiting a 90.7% recovery rate with a purity yield of 86.9%, with short processing time of less than 2 hours and low cost. By altering pore sizes of nanomembrane filters and optimizing experiment parameters, the platforms and methods presented herein are able to investigate separation of subgroups of exosomes and dissect their biological functions. This improved technique is able to serve as a standardized exosome separation tool, and to accelerate exosome studies to explore its clinical prospects within liquid biopsies. As a result, the present disclosure can open the avenue for the point-of-care applications of exosomes as prognostic and diagnostic biomarkers in the future. Additionally, one or more of the porous nanomembranes can be associated with a hydrogel with a surface potential (e.g., a positive surface potential) to assist in the microRNA extraction by utilizing methods where size-isolated exosomes are trapped at the hydrogel porous nanomembrane, then to extract microRNA in exosome, an exosome lysis buffer can be pumped into the second channel. It is further envisioned that more layers and porous nanomembranes having various porosities could be utilized to isolate particles having various sizes. Additionally, it is further envisioned that surface charges of the hydrogels, as disclosed herein, can be altered to attract or repel desired particles for isolation or exclusion.

### Additional Embodiments

The microfluidic devices of the present disclosure and the methods of use thereof can have numerous embodiments. For example, in an embodiment, the present disclosure relates to a microfluidic platform (e.g., a selective isolation platform) that includes a top layer having a top inlet and a top outlet, a center layer having a center inlet and a center outlet, a bottom layer having a bottom inlet and a bottom outlet, a first porous membrane between the top layer and the center layer, a second porous membrane between the center layer and the bottom layer, a first electrode disposed on at least one of the top layer and the bottom layer, and a second electrode disposed on at least one of the top layer and the bottom layer.

In some embodiments, the microfluidic platform (e.g., an isolation platform) further includes a hydrogel having a charge including a positive charge or a negative charge. In some embodiments, the hydrogel is disposed between the center layer and the bottom layer. In some embodiments, the second porous membrane is integrated with the hydrogel. In some embodiments, the microfluidic platform (e.g., an isolation platform) has a pre-concentration of exosomes via the hydrogel.

In some embodiments, the top inlet and the top outlet are in fluid communication. In some embodiments, the center inlet and the center outlet are in fluid communication. In some embodiments, the bottom inlet and the bottom outlet are in fluid communication. In some embodiments, the first porous membrane provides fluid communication between the top layer and the center layer. In some embodiments, the second porous membrane provides fluid communication between the center layer and the bottom layer.

In some embodiments, the microfluidic platform (e.g., isolation platform) further includes a second center layer having a second center inlet and a second center outlet and a third porous membrane between the center layer and the second center layer. In some embodiments, the second center inlet and the second center outlet are in fluid communication and the third porous membrane provides fluid communication between the center layer and the second center layer. In some embodiments, the microfluidic platform (e.g., an isolation platform) further includes a third center layer having a third center inlet and a third center outlet and a fourth porous membrane between the second center layer and the third center layer. In some embodiments, the third center inlet and the third center outlet are in fluid communication and the fourth porous membrane provides fluid communication between the second center layer and the third center layer.

In another embodiment, the present disclosure pertains to a method for selective isolation. In some embodiments, the method includes flowing a sample through an isolation platform (e.g., a microfluidic platform). In some embodiments, the isolation platform (e.g., a microfluidic platform) includes a top layer having a top inlet and a top outlet, a center layer having a center inlet and a center outlet, a bottom layer having a bottom inlet and a bottom outlet, a first porous membrane between the top layer and the center layer, a second porous membrane between the center layer and the bottom layer, a first electrode disposed on at least one of the top layer and the bottom layer, and a second electrode disposed on at least one of the top layer and the bottom layer.

In some embodiments, the top layer includes a first input port, a second input port, a first exit port, and a second exit port. In some embodiments, the center layer includes a first through port and a second through port. In some embodiments, the first input port is fluidly coupled to the center inlet such that fluid flows from the first input port to the center inlet. In some embodiments, the first exit port is fluidly coupled to the center outlet such that fluid flows from the center outlet to the first exit port. In some embodiments, second input port is fluidly coupled to the first through port which is fluidly coupled to the bottom inlet such that fluid flows from the first input port through the first through port and to the bottom inlet. In some embodiments, the second exit port is fluidly coupled to the second through port which is fluidly coupled to the bottom outlet such that fluid flows from the bottom outlet through the second through port and to the second exit port. In some embodiments, multiple input ports, exit ports, and through ports are readily envisioned for embodiments with multiple center layers.

In some embodiments, the method further includes isolating a first component from the sample in the top layer, where the first porous membrane precludes the first component from flowing into the center layer, isolating a second component from the sample in the center layer, where the second porous membrane precludes the second component from flowing into the bottom layer, and isolating a third component from the sample in the bottom layer.

In some embodiments, the isolation platform (e.g., a microfluidic platform) used for the method further includes a hydrogel having a charge including a positive charge or a negative charge. In some embodiments, the hydrogel is disposed between the center layer and the bottom layer. In some embodiments, the method further includes isolating a component from the sample in the center layer, where the charge of the hydrogel interacts with particles in the sample to attract the component, and pumping an exosome lysis buffer into a channel disposed in the center layer. In some embodiments, a flowrate of the exosome lysis buffer in the channel prevents trapping of microRNA in the sample. In some embodiments, the method further includes extracting the microRNA from the sample in the center layer. In some embodiments, the second porous membrane is integrated with the hydrogel. In some embodiments, the isolation platform (e.g., a microfluidic platform) has a pre-concentration of exosomes via the hydrogel.

In some embodiments, the first component includes large-sized particles. In some embodiments, the large-sized particles have a diameter greater than a porosity of the first porous membrane. In some embodiments, the second component includes medium-sized particles. In some embodiments, the medium-sized particles have a diameter less than a porosity of the first porous membrane and greater than a porosity of the second porous membrane. In some embodiments, the medium-sized particles include at least one of extracellular vesicles, exosomes, and liposomes. In some embodiments, the third component includes small-sized particles. In some embodiments, the small-sized particles have a diameter less than a porosity of the first porous membrane and a porosity of the second porous membrane. In some embodiments, the small-sized particles include, without limitation, proteins, RNA, DNA, RNA fragments, DNA fragments, microRNA, and combinations thereof.

In some embodiments, the method further includes identifying biomarkers in at least one of the first component, the second component, and the third component, and determining if a disease is present in a subject supplying the sample based, at least in part, on the identifying. In some embodiments, the disease includes, without limitation, cancer, diabetes, autoimmune diseases, Parkinson's disease, Alzheimer's, or combinations thereof.

In a further embodiment, the present disclosure pertains to a microfluidic platform (e.g., isolation platform) including a top layer having a top inlet and a top outlet, a plurality of center layers, each center layer of the plurality of center layers having a center inlet and a center outlet, a bottom layer having a bottom inlet and a bottom outlet, a first porous membrane between the top layer and a top center layer of the plurality of center layers, a plurality of center porous membranes between each center layer of the plurality of center layers, a second porous membrane between the bottom layer and a lowest center layer of the plurality of center layers, a first electrode disposed on at least one of the top layer and the bottom layer, and a second electrode disposed on at least one of the top layer and the bottom layer.

In some embodiments, the microfluidic platform (e.g., isolation platform) includes a hydrogel having a charge including a positive charge or a negative charge. In some embodiments, the hydrogel is disposed between any of the top layer, center layers, and the bottom layer. In some embodiments, at least one porous membrane including, but not limited to, the first porous membrane, the plurality of center porous membranes, and the second porous membrane is integrated with the hydrogel. In some embodiments, the microfluidic platform has a pre-concentration of exosomes via the hydrogel. In some embodiments, the top inlet and the top outlet are in fluid communication, and the bottom inlet and the bottom outlet are in fluid communication. In some embodiments, each center inlet and each center outlet of the plurality of center layers are in fluid communication. In some embodiments, the first porous membrane provides fluid communication between the top layer and the top center layer, and the second porous membrane provides fluid communication between the lowest center layer and the bottom layer. In some embodiments, each center porous membrane of the plurality of center porous membranes are in fluid communication with a lower center layer of the plurality of center layers.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1-25. (canceled)

26. A microfluidic platform comprising:
a top layer comprising a top inlet and a top outlet;
a plurality of center layers, each center layer of the plurality of center layers comprising a center inlet and a center outlet;
a bottom layer comprising a bottom inlet and a bottom outlet;
a first porous membrane between the top layer and a top center layer of the plurality of center layers;
a plurality of center porous membranes between each center layer of the plurality of center layers;
a second porous membrane between the bottom layer and a lowest center layer of the plurality of center layers;
a first electrode disposed on at least one of the top layer and the bottom layer; and
a second electrode disposed on at least one of the top layer and the bottom layer.

27. The microfluidic platform of claim 26, comprising a hydrogel having a charge selected from the group consisting of a positive charge and a negative charge, wherein the hydrogel is disposed between a layer selected from the group consisting of the top layer, at least one center layer of the plurality of center layers, and the bottom layer.

28. The microfluidic platform of claim 27, wherein at least one porous membrane selected from the group consisting of the first porous membrane, the plurality of center porous membranes, and the second porous membrane is integrated with the hydrogel.

29. The microfluidic platform of claim 27, wherein the microfluidic platform has a pre-concentration of exosomes via the hydrogel.

30. The microfluidic platform of claim 26, wherein the top inlet and the top outlet are in fluid communication, and wherein the bottom inlet and the bottom outlet are in fluid communication.

31. The microfluidic platform of claim 26, wherein each center inlet and each center outlet of the plurality of center layers are in fluid communication.

32. The microfluidic platform of claim 26, wherein the first porous membrane provides fluid communication between the top layer and the top center layer, and wherein the second porous membrane provides fluid communication between the lowest center layer and the bottom layer.

33. The microfluidic platform of claim 26, wherein each center porous membrane of the plurality of center porous membranes are in fluid communication with a lower center layer of the plurality of center layers.

34. The microfluidic platform of claim 26, comprising a hydrogel disposed between a layer selected from the group consisting of the top layer, at least one center layer of the plurality of center layers, and the bottom layer.

35. The microfluidic platform of claim 34, wherein the hydrogel comprises polyethylene glycol dimethacrylate (PEGDA)-tri.

36. The microfluidic platform of claim 35, wherein the PEGDA-tri comprises a PEGDA backboard and amine group at its molecular terminal.

37. The microfluidic platform of claim 34, wherein the hydrogel is positively charged to attract negatively charged exosomes.

38. The microfluidic platform of claim 34, wherein the hydrogel has a pH under 7.

39. The microfluidic platform of claim 34, wherein at least one of the first porous membrane or the second porous membrane has a 30 nm porosity.

40. The microfluidic platform of claim 34, wherein at least one of the first porous membrane or the second porous membrane is integrated with a polyethylene glycol dimethacrylate-triamine (PEGDA-triA) hydrogel.

41. The microfluidic platform of claim 40, wherein the first porous membrane has a pore size of 100 nm and the second porous membrane has a pore size of 30 nm.

42. The microfluidic platform of claim 40, wherein the at least one of the first porous membrane or the second porous membrane has a positive surface potential to attract negatively charged molecules.

43. The microfluidic platform of claim 26, wherein at least one center porous membrane of the plurality of center porous membranes is sized to filter micro vesicles.

44. The microfluidic platform of claim 26, wherein at least one center porous membrane of the plurality of center porous membranes is sized to filter extracellular vesicles.

45. The microfluidic platform of claim 26, wherein at least one center porous membrane of the plurality of center porous membranes is sized to filter at least one of protein, RNA, or DNA fragments.
